(19) United States
(12) Patent Application Publication
(10) Pub. No.: US 2025/0073441 A1
(43) Pub. Date: Mar. 6, 2025

(54) CONNECTOR ASSEMBLY FOR FLUID PORTS

(71) Applicant: Alcon Inc., Fribourg (CH)

(72) Inventors: Parthasarathy PARISHRAM, Irvine, CA (US); Kyle Randall LEONG, Tustin, CA (US)

(21) Appl. No.: 18/812,778

(22) Filed: Aug. 22, 2024

Related U.S. Application Data
(60) Provisional application No. 63/579,124, filed on Aug. 28, 2023.

(51) Int. Cl. A61M 39/10; A61F 9/007

(52) U.S. Cl. CPC A61M 39/1011; A61B 2217/005; A61F 9/00736; A61M 2202/02; A61M 2205/3306; A61M 2205/587; A61M 2210/0612

(57) ABSTRACT

The present disclosure generally relates to port connector assemblies for delivering infusion gases during ophthalmic surgeries and procedures. In some embodiments, the connector assembly includes a connector for a surgical console and a corresponding receiver for connecting the connector to the surgical console.

### CROSS-REFERENCE TO RELATED APPLICATIONS

This application claims priority to U.S. Provisional Application No. 63/579,124, filed on Aug. 28, 2023, which is hereby incorporated by reference in its entirety.

### INTRODUCTION

During ophthalmic surgical procedures, liquids and/or other fluids, such as saline solution and/or air, are often supplied to a patient's eye through one or more supply lines fluidically coupled to a surgical console. To ensure sterility between surgical procedures, new supply lines are coupled to the surgical console for each procedure and subsequently discarded after use.

### SUMMARY

The present disclosure generally relates to devices for ophthalmic procedures, and more specifically, to devices for delivering infusion gases. In some embodiments, a connector for a surgical console is provided. The connector includes a proximal portion having a proximal port configured to be received by the surgical console, and a distal portion having a distal port. The connector also includes a tube extending between the proximal portion and the distal portion, the proximal port in fluid communication with the distal port via the tube and the tube having a flow axis extending along and substantially parallel with a direction of flow between the proximal port and the distal port. An annular flange formed along an outer surface of the tube and extending perpendicular to the flow axis from the outer surface of the tube partitions the proximal portion from the distal portion. The proximal portion of the connector includes one or more light posts extending from the proximal port. Each of the one or more light posts extends parallel to the flow axis of the tube and is configured to interact with a light pipe of the surgical console when the proximal portion of the connector is connected to the surgical console.

In another embodiment, a receiver for connecting a connector to a surgical console is provided. The receiver includes a port disposed at a distal end of the receiver opposite of a proximal end of the receiver. The port at the distal end of the receiver is sized and shaped to receive the connector, and the proximal end of the receiver is configured to connect to the surgical console. The receiver also includes a tube extending through the receiver from the port at the distal end of the receiver to the proximal end and pair of light pipe apertures. The tube has a flow axis extending along and substantially parallel with a direction of flow through the port and the tube. The light pipe apertures are configured to receive an extension of a light pipe through the tube, the light pipe extending substantially perpendicular with the flow axis of the tube.

In further embodiments, a connector assembly for a surgical console is provided. The assembly includes a receiver sized and shaped to be received by a receiver portion in the surgical console, and a connector sized and shaped to be inserted into the receiver. The receiver portion of the surgical console is in fluid communication with an infusion or vacuum gas. The receiver includes a port disposed at a distal end of the receiver opposite of a proximal end of the receiver. The port at the distal end of the receiver is sized and shaped to receive the connector, and the proximal end of the receiver is configured to connect to the surgical console. The receiver also includes a receiver tube extending through the receiver from the port at the distal end of the receiver to the proximal end. The receiver has a flow axis extending along and substantially parallel with a direction of flow through the port and the receiver tube. The receiver also includes a pair of light pipe apertures configured to receive extension of a light pipe through the receiver tube, the light pipe extending substantially perpendicular with the flow axis of the receiver.

The connector of the assembly includes a proximal portion having a proximal port configured to be received by the surgical console, and a distal portion having a distal port. The connector also includes a connector tube extending between the proximal portion and the distal portion, the proximal port in fluid communication with the distal port via the connector tube and the connector tube having a flow axis extending along and parallel with a direction of flow between the proximal port and the distal port. An annular flange formed along an outer surface of the connector tube and extending perpendicular to the flow axis from the outer surface of the connector tube partitions the proximal portion from the distal portion. The proximal portion of the connector includes one or more light posts extending from the proximal port. The one or more light posts are configured to obstruct the extension of the light pipe through the receiver tube when the receiver is received by the surgical console and the proximal portion of the connector is inserted into the receiver and rotated. When the receiver is received by the receiver portion of the surgical console and the connector is coupled with the receiver, the proximal port of the connector is in fluid communication with the infusion or vacuum gas source.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates a perspective view of an exemplary surgical console, according to certain embodiments;

FIGS. 2A and 2B illustrate perspective views of an exemplary connector assembly, including an exemplary connector and an exemplary receiver, for use with the surgical console of FIG. 1, according to certain embodiments;

FIGS. 3A and 3B illustrate perspective views of the connector of FIGS. 2A and 2B, according to certain embodiments;

FIG. 3C illustrates a schematic cross-sectional view of the connector of FIGS. 3A and 3B, according to certain embodiments;

FIG. 3D illustrates an enlarged perspective view of a portion of the connector of FIGS. 3A and 3B, according to certain embodiments;

FIGS. 3E and 3F illustrate end views of the connector of FIGS. 3A and 3B, according to certain embodiments;

FIGS. 4A-4C illustrate perspective, frontal, and schematic cross-sectional views, respectively, of the receiver of FIGS. 2A and 2B, according to certain embodiments;

FIGS. 5A-5C illustrate the connector assembly of FIGS. 2A and 2B, during use, according to certain embodiments;

FIGS. 6A and 6B illustrate schematic cross-sectional views of the connector assembly, during use, as seen from section lines 6A-6A and 6B-6B in FIGS. 5B and 5C, respectively, according to certain embodiments;

FIGS. 7A and 7B illustrate perspective cross-sectional views of the connector assembly, during use, according to certain embodiments; and

FIG. 7C illustrates a schematic cross-sectional view of connector assembly during use, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end or portion of a component refers to the end or portion that is closer in line to a patient's body during use thereof. On the other hand, a proximal end or portion of the component refers to the end or the portion that is distanced further away in line from the patient's body (e.g., closer to the surgical console).

When the word “substantially” is used, this term may mean that there may be a variance in value of up to ±10%, of up to 5%, of up to 2%, of up to 1%. For example, “substantially parallel” or “substantially perpendicular” means there may be a variance in the orientation between two objects of ±1-5 degrees.

To ensure patient safety and efficient performance of a given ophthalmic surgical procedure, the fluid supply lines must be properly connected to the surgical console. Otherwise, the flow and pressure of such fluids through the supply lines may be inconsistent and negatively affect the performance of the procedure. An improper connection of a supply line may also cause the supply line to inadvertently disconnect from the surgical console during the procedure, thereby interrupting the flow of fluids from the surgical console and create an increased safety risk for the patient. Accordingly, what is needed in the art are improved connectors for connecting fluid supply lines to surgical consoles.

The present disclosure generally relates to port connector assemblies for connecting fluid supply lines used for delivering infusion gases during ophthalmic surgeries and procedures. The following description provides a connector assembly for removably, but securely, connecting a gas supply line to a supply port of a surgical console and/or other gas source. The connector assembly includes a connector configured to couple to a proximal end of the gas supply line, and a corresponding receiver configured to operably couple to a surgical console and receive the connector. In some embodiments, the described connector assembly facilitates reduced direct handling of the gas supply line itself, as only the connector and receiver need be engaged/handled by the user when connecting the new gas supply line. This helps maintain the sterility and integrity of the gas supply line, as well as facilitate efficient connecting and disconnecting of the line from the surgical console before and after each procedure.

In some embodiments, the described connector assembly also facilitates secure temporary locking between the connector and corresponding receiver/port, as well as provides indications to the surgical console when said connector and corresponding receiver/port are in a locked state. The connector assembly can therefore help the surgeon confirm the gas supply line is properly and securely connected prior to starting the surgery, as well as prevents the connector from inadvertently disconnecting from the corresponding receiver/port during the surgery.

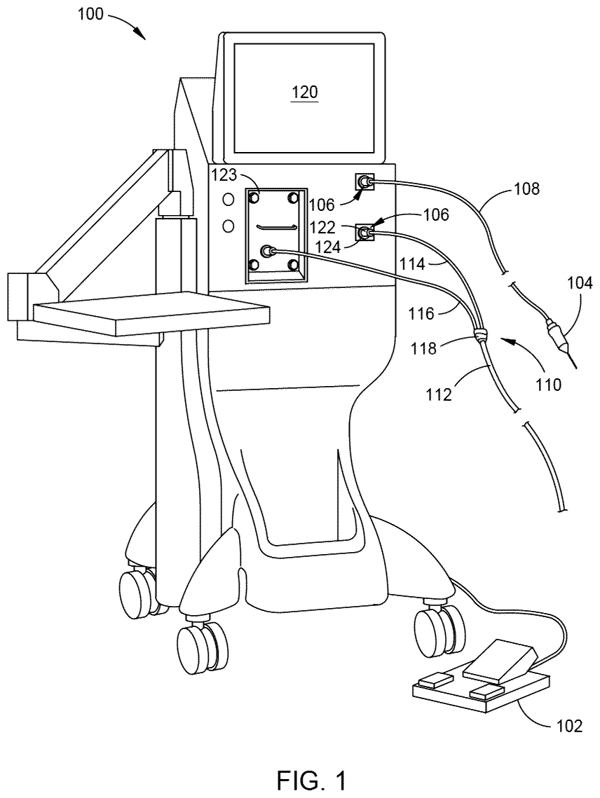

FIG. 1 illustrates a perspective view of an exemplary surgical console 100 for which connector assembly described herein may be utilized in combination with. Surgical console 100 is operably coupled, physically or wirelessly, to any number of user interfaces, including a foot controller 102 and a surgical tool 104, such as a vitrectomy probe or other device. Surgical console 100 provides one or more ports 106 for physically coupling the user interfaces to various components of surgical console 100. For example, surgical tool 104 may be fluidly coupled with a vacuum source via a supply line 108 connected to a port 106 to enable aspiration of cut vitreous from the patient's eye. However, supply line 108 and port 106 may also be representative of other types of supply lines and/or ports, respectively, for coupling with surgical tool 104 as needed during a given surgical procedure.

Similarly, one or more other ports of surgical console 100 may be utilized to connect a fluid infusion system with one or more infusion fluid sources, (e.g., an air/gas source, a liquid perfluorocarbon source, a silicone oil infusion (SOI) source, a BSS (Balanced Salt Solution) source, etc.) to enable infusion of fluids into the eye during vitreous removal. As shown in FIG. 1, exemplary fluid infusion system 110 includes an infusion line 112 fluidly coupled with a gas supply line 114 and a liquid supply line 116 at a three-way automatic valve assembly 118, which may enable selective flow of different infusion fluids and/or compositions through infusion line 112. Gas supply line 114 may be fluidly coupled with a gas source via port 106 of surgical console 100. Liquid supply line 116, on the other hand, may be fluidly coupled with a liquid source through ports in a surgical cassette 123 used with surgical console 100. In certain other embodiments, liquid supply line 116 may alternatively be coupled with the liquid source and surgical console 100 directly via another port 106 of surgical console 100. Generally, the connection of each supply line to the surgical cassette 123 or surgical console 100 is accomplished via mating of a connector attached to the supply line and a corresponding receiver on the cassette or console. Together, such connectors and their corresponding receivers may be referred to herein as “connector assemblies.” As shown in FIG. 1, a connector 122 is used to connect gas supply line 114 to a corresponding receiver 124 of port 106 of surgical console 100.

In operation, a user may control an aspect or mechanism of surgical tool 104 and/or fluid infusion system 110 via actuation of the foot controller 102. For example, the user may press down on (e.g., depress) the foot controller 102 to initiate and/or increase a flow rate of an infusion fluid from a fluid source through fluid infusion system 110 and into the eye of the patient. Alternatively, reducing depression of foot controller 102 (e.g., lifting the user's foot) may decrease and ultimately stop the flow of fluid through fluid infusion system 110. Accordingly, in certain embodiments, the flow rate of infusion fluids through fluid infusion system 110 corresponds to the amount of depression of foot controller 102. In further embodiments, the user may select between fluid sources using foot controller 102.

In certain embodiments, surgical console 100 further includes a display 120 for displaying information to the user (the display may also incorporate a touchscreen for receiving user input). Thus, display 120 may display information about each of the user interfaces connected to surgical console 100. For example, with reference to fluid infusion system 110, display 120 may show information related to infusion fluid parameters (e.g., infusion fluid flow rates and intraocular pressure) to the user during operation thereof.

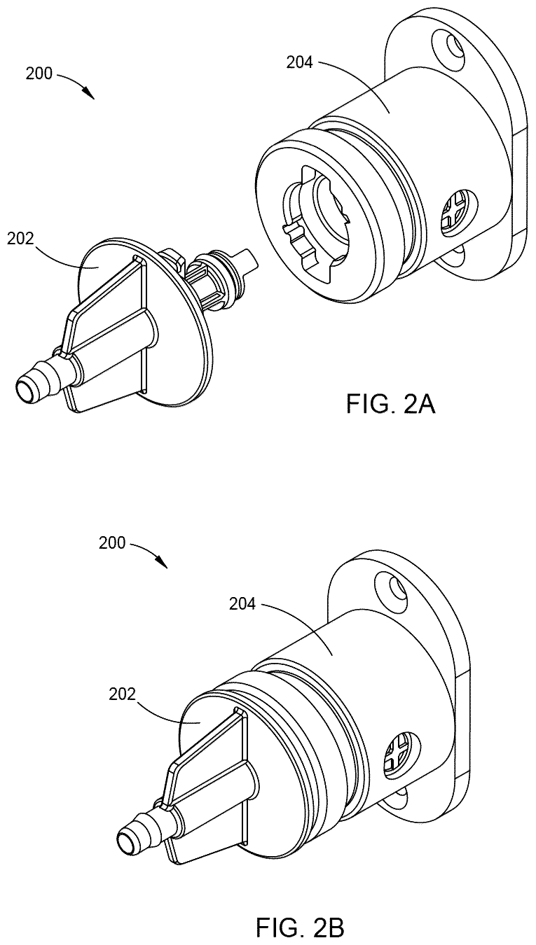

FIGS. 2A and 2B illustrate a connector assembly 200 that may be implemented with surgical console 100 for removably connecting gas supply line 114 with a gas source. As shown in FIGS. 2A and 2B, connector assembly 200 generally includes a connector 202 configured to removably couple with a corresponding receiver 204 (hereafter “receiver 204”). Connector 202 and receiver 204 are examples of connector 122 and receiver 124 shown in FIG. 1.

Generally, connector 202 removably couples with receiver 204 on one end of connector 202 and removably connects to gas supply line 114 on an opposite end thereof. Meanwhile, receiver 204 can be fixedly integrated with surgical console 100 at port 106 for receiving connector 202, or receiver 204 can be removably coupled with surgical console 100 at port 106. When connector 202 is coupled to receiver 204, as shown in FIG. 2A, connector assembly 200 provides a fluidly sealed tube for fluidly coupling gas supply line 114 to port 106. During use, the tube extending through connector assembly 200 enables bi-directional flow of gases, such as low pressure air, between gas supply line 114 and a gas source connected with the surgical console 100. The connector 202 may also be disconnected from receiver 204, as shown in FIG. 2B, to disconnect gas supply line 114 from corresponding receiver 204, and thus, port 106 of the surgical console 100.

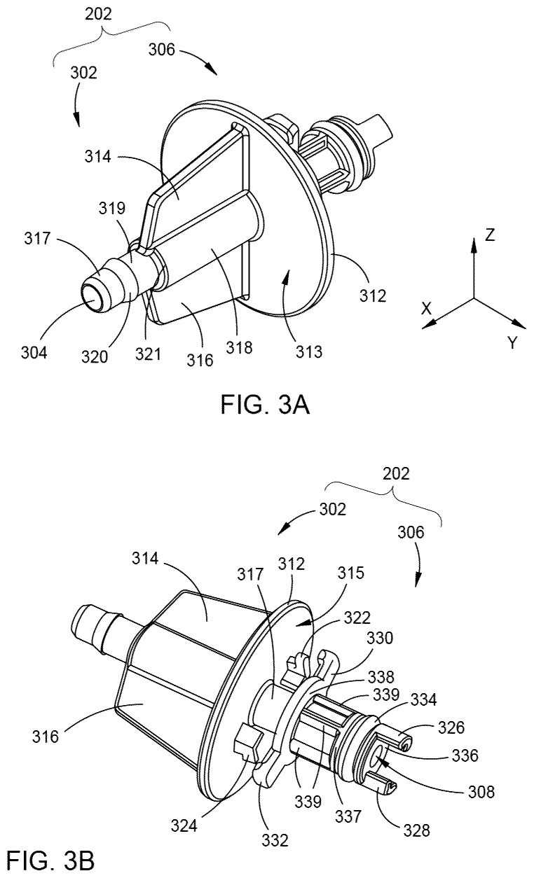

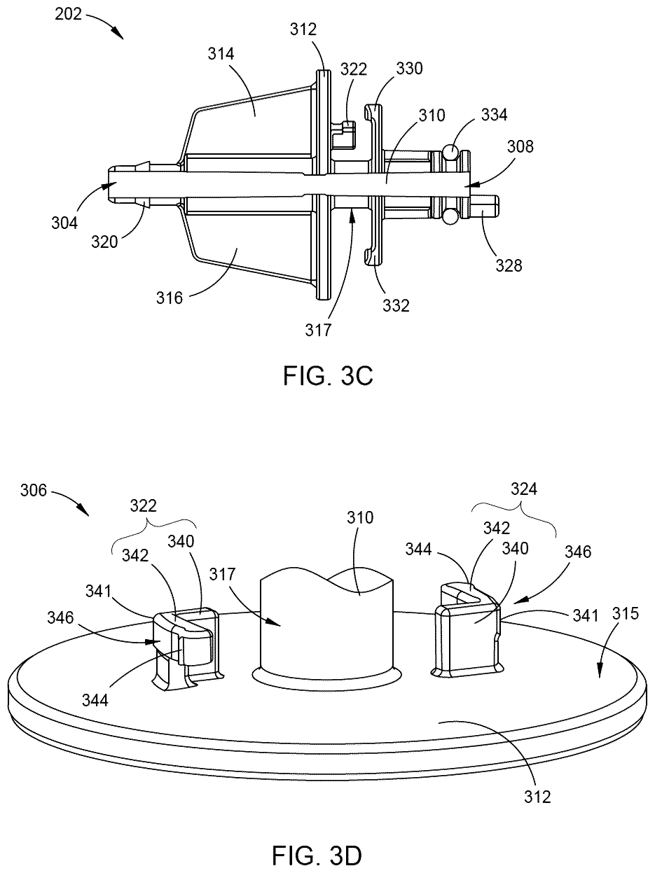

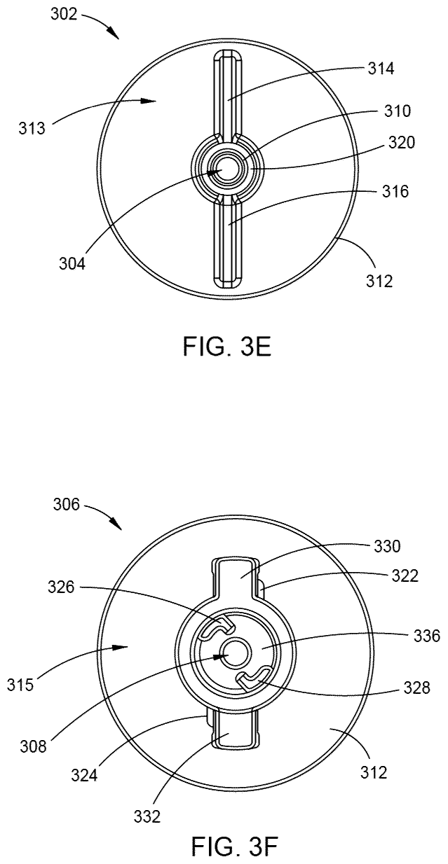

Turning now to FIGS. 3A-3E, FIGS. 3A and 3B illustrate perspective views of an exemplary connector 202, according to certain embodiments; FIG. 3C illustrates a cross-sectional view of connector 202, according to certain embodiments; FIG. 3D illustrates an enlarged perspective view of a portion of connector 202, according to certain embodiments; and FIGS. 3E-3F illustrate end views of connector 202, according to certain embodiments. Accordingly, FIGS. 3A-3E are herein described together for clarity.

Connector 202 generally includes a distal portion 302 having a distal port 304 that is disposed opposite of a proximal portion 306 having a proximal port 308. Distal port 304 is in fluid communication with proximal port 308 via a connector tube 310 (shown in FIG. 3C) extending through connector 202. An annular flange 312 having a distal side 313 and a proximal side 315 is disposed between the distal portion 302 and the proximal portion 306. Annular flange 312 extends radially from an exterior surface 317 of connector tube 310 in a manner substantially perpendicular to a longitudinal flow axis of connector tube 310. That is, a major plane of the annular flange 312 is substantially perpendicular to the longitudinal flow axis of connector tube 310. Annular flange 312 generally divides distal portion 302 of connector 202 from proximal portion 306 with distal side 313 of annular flange 312 facing the distal portion 302 of connector 202 and proximal side 315 facing proximal portion 306. When connector 202 is in use, annular flange 312 may provide protection against user contact with non-sterile surfaces of surgical console 100 as distal portion 302 is handled and manipulated to connect proximal portion 306 of connector 202 with port 106 of surgical console 100 or receiver 204 disposed therein. The connector 202 may be made from any materials commonly used for such connectors and suitable for ophthalmic surgical settings. For example, the connector 202 may be formed of thermoplastic polymers and other polymeric materials, lightweight aluminum, anodized aluminum, stainless steel and other metallic alloys, or other suitable materials.

As shown in FIG. 3A, distal portion 302 of connector 202 also includes a first fin 314, a second fin 316, a tube member 318, and a barbed collar 320. First and second fins 314, 316 extend from an exterior surface of tube member 318. First and second fins 314, 316 also extend from the distal side of annular flange 312 such that the first and second fins 314, 316 are oriented perpendicular to a major plane of the annular flange 312. During use, first and second fins 314, 316 enable gripping and manipulating (e.g., rotating/turning) of connector 202 by the user for connecting connector 202. Barbed collar 320 is disposed between distal port 304 and a distal end 321 of tube member 318. In some embodiments, first fin 314, second fin 316, tube member 318, and barbed collar 320 may be formed as a single unitary component of connector 202, for example, such as by injection molding. In some embodiments, first fin 314, second fin 316, tube member 318, and barbed collar 320 may each be separate parts assembled together for connector 202. In such embodiments, barbed collar 320 can assist in retaining, via friction, tubes (e.g., tube member 318) disposed over an exterior surface 319 of connector tube 310.

During use, barbed collar 320 facilitates mechanical retention of a flexible tube (e.g., gas supply line 114) disposed over distal port 304. For example, barbed collar 320 may be sized to be greater than a proximal end of the flexible tubing of gas supply line 114 such that the proximal end retracts against the barbed collar 320 when stretched over the distal port 304 and the barbed collar 320. The tension from the retraction of the tubing against the barbed collar 320, in turn, applies a mechanical force for retaining the proximal end of the tubing of the gas supply line 114 over distal port 304 and connector tube 310.

In certain other embodiments, distal portion 302 of connector 202 may not include barbed collar 320. Instead, an adhesive can alternatively be used to connect and seal the gas supply line 114 and distal port 304 of connector 202 together. For example, an adhesive can be applied between an interior surface of tubing for gas supply line 114 and the exterior surface 317 of connector tube 310 to connect the tubing over distal port 304.

In another embodiment, connector 202 may alternatively be inserted into distal port 304 to connect gas supply line 114 and connector 202 together. For example, distal port 304 and connector tube 310 may be configured with a larger interior diameter such that a proximal end of tubing for gas supply line 114 can be inserted into distal port 304 and secured along an interior surface of connector tube 310. In such embodiments, an adhesive can be used between an exterior surface of the tubing at the proximal end of gas supply line 114 and the interior surface of connector tube 310 to seal and bond the tubing of gas supply line 114 and connector 202 together.

Turning to FIGS. 3B and 3D, proximal portion 306 includes a first locking tab 322 and second locking tab 324 extending from proximal side 315 of annular flange 312. Generally, first and second locking tabs 322, 324 enable connector 202 to mate and lock with receiver 204 when the two are connected, as described in more detail below. In certain embodiments, first and second locking tabs 322, 324 are each formed as angled structures disposed on opposite sides of connector tube 310, as shown in FIG. 3D. First and second locking tabs 322, 324, may each include a first member 340 connected to a second member 342 at a non-zero angle, thus forming a bend 341, with first member 340 extending towards the connector tube 310 and second member 342 extending substantially perpendicular relative to first member 340. Second member 342 has an increasing lateral thickness along a length thereof that starts from the bend 341 adjacent to first member 340 and terminates at a ledge 344 disposed furthest from first member 340. Ledge 344 may generally include an overhang that extends from an end of second member 342 opposite of bend 341 towards an outer surface 346 of second member 342 facing away from the connector tube 310. Second member 342 and ledge 344 are configured to flex towards connector tube 310 in response to a force applied against outer surface 346 of second member 342.

As shown in FIG. 3B, proximal portion 306 further includes a first light post 326, a second light post 328, a first aligner 330, a second aligner 332, and a removable o-ring 334 disposed along the exterior surface 317 of connector tube 310. First and second aligners 330, 332 extend from a ring 338 disposed adjacent to the first and second locking tabs 322, 324 and annular flange 312. Ring 338 extends from exterior surface 317 of connector tube 310 and is oriented substantially parallel with annular flange 312. First and second aligners 330, 332 are disposed on opposite sides of ring 338 and each extend substantially perpendicular to the longitudinal flow axis of connector tube 310, e.g., parallel with the X axis in FIG. 3B. Ring 338 is further connected to a collar 337 disposed proximal to ring 338 and closer to proximal port 308. Ring 338 is connected to collar 337 by a plurality of support members 339 extending along the exterior surface 317 of connector tube 310 parallel with the longitudinal flow axis of connector tube 310.

First and second light posts 326, 328 extend from a proximal end 336 of connector tube 310 in an orientation substantially parallel with the longitudinal flow axis of connector tube 310. As shown in FIGS. 3B and 3F, the first and second light posts 326, 328 are disposed on opposite sides of proximal port 308 and each have an arcuate shape. As shown in FIGS. 3B and 3C, proximal portion 306 includes a channel formed between collar 337 and a base of first and second light posts 326, 328 near proximal end 336. O-ring 334 is disposed in the channel with collar 337 on one side of o-ring 334 and first and second light posts 326, 328 on the other side thereof. As discussed in more detail below, o-ring 334 may extend around the exterior surface 317 of connector tube 310 and be used to create a seal in a portion of receiver 204 when connector 202 is inserted into receiver 204.

FIGS. 3E and 3F show end views of connector 202. Turning to FIG. 3E, first and second fins 314, 316 are disposed on opposite sides of distal port 304 and connector tube 310. As shown in FIGS. 3B and 3F, first and second aligners 330, 332 are aligned with first and second locking tabs 322, 324 disposed on annular flange 312, respectively, on opposite sides of connector tube 310. First and second aligners 330, 332 are therefore each disposed across from first and second locking tabs 322, 324, such that first and second aligners 330, 332 are similarly also disposed on opposite sides of connector tube 310.

As described in more detail below, first and second aligners 330, 332 ensure the orientation of connector 202 is proper when connector 202 is connected to receiver 204 by the user. Although first and second aligners 330, 332 are shown disposed on opposite sides of connector tube 310 (e.g., 180 degrees opposite of one another), first aligner 330 and second aligner 332 may also be disposed in alternative placements relative to one another. For example, in certain other embodiments, first and second aligners 330, 332 may extend perpendicular to one another with first and second locking tabs 322, 324 correspondingly aligned. In further embodiments, connector 202 may include just one aligner or more than two aligners with receiver 204 similarly configured to receive the corresponding connector 202 thereof. In further embodiments, connector 202 may also include just one locking tab or more than two locking tabs with receiver 204 similarly configured for locking connector 202 and corresponding receiver 204 together.

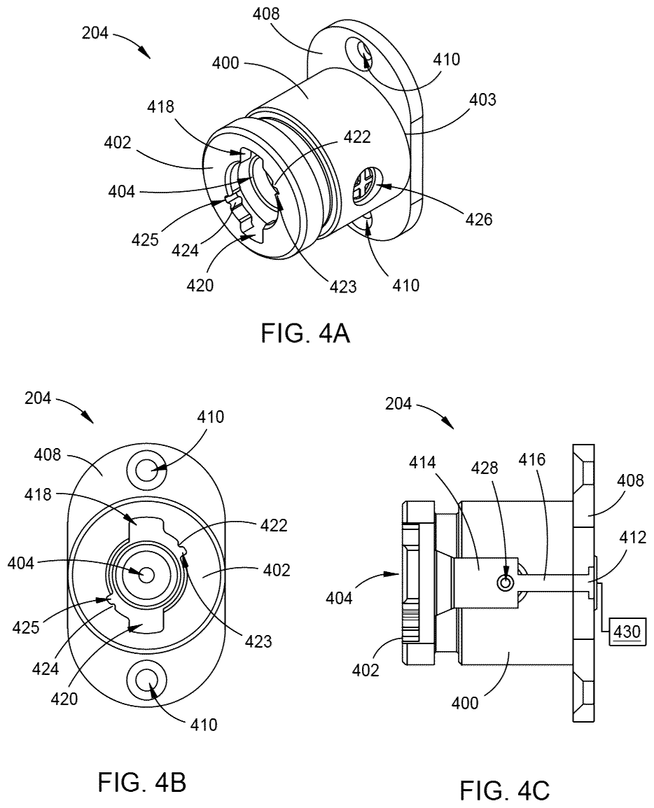

FIGS. 4A-4C illustrate perspective and cross-sectional views of an exemplary receiver 204, according to certain embodiments. Receiver 204 generally includes a housing 400 having a receiver port 404 at a distal end 402 and a base 408 disposed at a proximal end 403. Receiver port 404 is configured to receive proximal portion 306 of connector 202 when coupling the connector 202 to receiver 204. Base 408 includes a pair of bores 410 to facilitate attachment and/or integration of receiver 204 with surgical console 100 at port 106. Proximal end 403 of receiver 204 also includes a supply port 412 extending through base 408. The receiver 204 may be made from any materials commonly used for such receivers and suitable for use with ophthalmic surgical consoles. For example, the receiver 204 may be formed of thermoplastic polymers and other polymeric materials, lightweight aluminum, anodized aluminum, stainless steel and other metallic alloys, or other suitable material. When receiver 204 is integrated with a surgical console, such as surgical console 100, supply port 412 may be in fluid communication with an infusion gas source 430 integrated with or coupled to the console.

As shown in FIG. 4C, receiver port 404 is also in fluid communication with supply port 412 via a chamber 414 and a receiver tube 416 extending through receiver 204. When connector 202 is connected with receiver 204, connector tube 310 of connector 202 is brought into fluid communication with receiver tube 416 of receiver 204, thereby enabling bi-directional flow of gases, such as low pressure air, between gas supply line 114 and infusion gas source 430, through connector 202 and receiver 204. In certain other embodiments, supply port 412 integrated with surgical console 100 may alternatively or additionally be in fluid communication with a vacuum source.

The size and shape of receiver port 404 of receiver 204 may be arranged to accommodate the proximal portion 306 of connector 202, including first and second aligners 330, 332. In other words, the receiver port 404 may have a morphology that substantially corresponds with the morphology of the proximal portion 306, such that the proximal portion 306 fits within the receiver port 404 of receiver 204 with a desired tolerance. In some examples, coupling connector 202 with receiver 204 also includes aligning one or more mating/engaging features therebetween. In certain embodiments, receiver port 404 may include a first aligner notch 418 opposite a second aligner notch 420 each sized and shaped for receiving corresponding first and second aligners 330, 332 of connector 202. Receiver port 404 may therefore be sized to enable proximal portion 306 of connector 202 to be fitted through receiver port 404 when first and second aligners 330, 332 of connector 202 are aligned with first and second aligner notches 418, 420. When connector 202 and receiver 204 are in use, the aligning of first and second aligners 330, 332 with the first and second aligner notches 418, 420 may therefore also facilitate the proper orientation of connector 202 when inserting connector 202 into the receiver 204.

An interior surface of receiver port 404 may also include a first locking protrusion 422, a second locking protrusion 424, a first locking notch 423, and a second locking notch 425. First locking protrusion 422 is disposed adjacent to first locking notch 423 and opposite second locking protrusion 424. Second locking notch 425 is disposed adjacent to second locking protrusion 424 and opposite first locking notch 423. As described in more detail below, first and second locking protrusions 422, 424 and first and second locking notches 423, 425 adjacent thereto, respectively, are configured to mate with first and second locking tabs 322, 324 of connector 202, respectively, to temporarily lock connector 202 and receiver 204 together when connected.

Housing 400 also includes a first light pipe aperture 426 opposite a second light pipe aperture 428. First and second light pipe apertures 426, 428 are configured to receive light through receiver 204 as propagated by a light pipe from an external environment, such as surgical console 100 when receiver 204 is integrated therein for port 106. As described in additional details below, first and second light pipe apertures 426, 428 enables surgical console 100 to propagate light through chamber 414 of receiver 204 for use in detecting proper connection between connector 202 and receiver 204 when in use. First and second light pipe apertures 426, 428 are disposed towards the proximal end of chamber 414 to enable the light propagating therethrough to be used to detect when the connector 202 is securely and fully inserted into receiver 204.

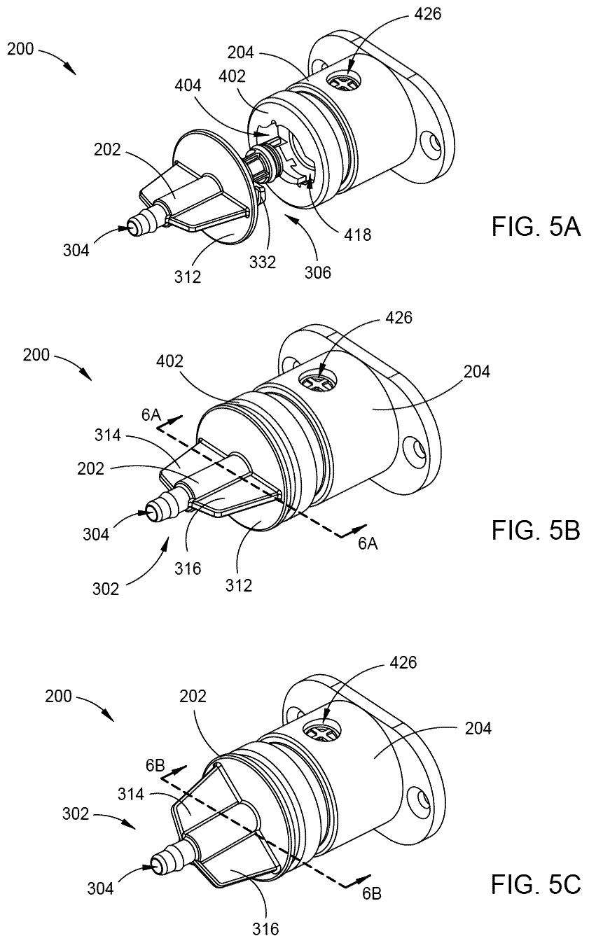

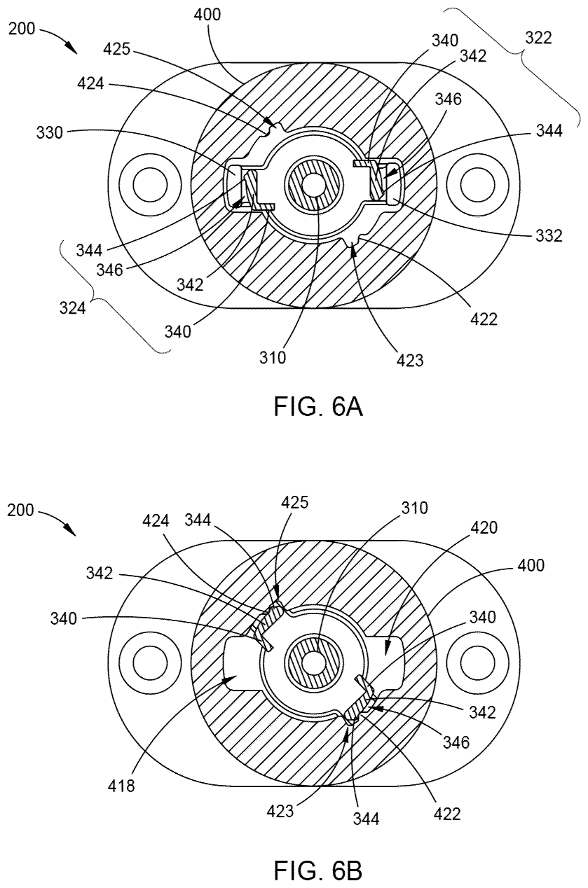

FIGS. 5A-5C illustrate perspective views of connector assembly 200 depicted in FIGS. 2A and 2B in use, according to certain embodiments. FIGS. 6A and 6B illustrate cross-sectional views of connector assembly 200 as seen from section lines 6A-6A and 6B-6B in FIGS. 5B and 5C, respectively. Accordingly, FIGS. 5A-5C and 6A and 6B are herein described together for clarity.

In operation, connector 202 is connected to receiver 204 by inserting proximal portion 306 of connector 202 into receiver port 404. As shown in FIG. 5A, to assist in proper orientation of connector 202 when first inserting proximal portion 306 thereof, first and second aligners 330, 332 of connector 202 are aligned with first and second aligner notches 418, 420 of receiver port 404 to enable proximal portion 306 of connector 202 to be fitted and inserted through receiver port 404. When proximal portion 306 of connector 202 is disposed within receiver port 404, annular flange 312 of connector 202 is brought adjacent to distal end 402 of receiver 204, as shown in FIG. 5B. When connector 202 is inserted into receiver 204, proximal portion 306, including proximal port 308 and first and second light posts 326, 328, extends into chamber 414. Proximal ends of first and second light posts 326, 328 in turn are disposed adjacent to a distal end of receiver tube 416.

O-ring 334 adjacent to first and second light posts 326, 328 is also disposed within chamber 414 when connector 202 is inserted into receiver 204. In certain embodiments, o-ring 334 comprises a gasket configured to create a seal in chamber 414 in the space between proximal port 308 and the distal end of receiver tube 416 when connector tube 310 is disposed in chamber 414. First and second light pipe apertures 426, 428 may each also be configured with an o-ring or other seal for further sealing chamber 414 of receiver 204. The seal created by o-ring 334 and any other o-rings in chamber 414 may assist in preventing gas flowing between connector tube 310 and receiver tube 416 from leaking out of chamber 414. O-ring 334 may be made from any materials commonly used for such gaskets for forming a seal between connecting components. For example, o-ring 334 may be formed of rubbers such as natural rubbers, neoprene rubbers, nitrile rubbers, and the like. A lubricant may also be applied with the o-ring 334 to assist in forming the seal and the inserting and withdrawing of the connector 202 from the receiver 204.

In some embodiments, as shown in FIGS. 5B and 5C, connector assembly 200 includes a locking mechanism in which connector 202 is rotated upon insertion of proximal portion 306 into receiver 204 to temporarily lock connector 202 and receiver 204 together. When connector assembly 200 is in such a locked state, connector assembly 200, in turn, requires an intentional counter rotation of connector 202 by the user to unlock and disconnect connector 202 from receiver 204. The locking mechanism therefore assists in maintaining the connection between connector 202 and receiver 204 during surgical procedures by preventing connector 202 and gas supply line 114 connected thereto from inadvertently disconnecting mid-procedure.

For example, as shown in FIG. 6A, when connector 202 is inserted into receiver 204 as shown in FIG. 5B, connector assembly 200 is initially in an unlocked state in which connector 202 can be freely translated through receiver port 404. In such an unlocked state, the first and second locking tabs 322, 324 of connector 202 are still aligned within first and second aligner notches 418, 420 due to the initial alignment of the first and second aligners 330, 332 needed for insertion of connector 202 through receiver port 404. Upon insertion of proximal portion 306 of connector 202 into receiver port 404, connector 202 can be rotated, as shown in FIG. 5C (e.g., using first and second fins 314, 316 extending from distal portion 302) to transition connector assembly 200 from the unlocked state to the locked state to temporarily lock connector 202 and receiver 204 together.

Turning to FIG. 6B, when connector 202 is rotated, the rotation of connector 202 correspondingly also rotates and moves first and second locking tabs 322, 324 of connector 202 along an interior surface of receiver port 404. In the example shown in FIG. 6A, due to the shape of receiver port 404, connector 202 is limited to being rotated clockwise to move first and second locking tabs 322, 324 towards first and second locking notches 423, 425, respectively.

As first and second locking tabs 322, 324 move along the interior surface of receiver port 404 during insertion, connector 202 may be rotated until each of the second members 342 of first and second locking tabs 322, 324 contact first and second locking protrusions 422, 424 and engage with first and second locking notches 423, 425, respectively. First and second locking protrusions 422, 424 may be shaped and sized to initially hinder further movement of first and second locking tabs 322, 324 as the ledge 344 of first and second locking tabs 322, 324 initially makes contact during rotation. However, with the application of sufficient rotational force by the user, the second members 342 of first and second locking tabs 322, 324 may each flex as the user urges the ledges 344 of first and second locking tabs 322, 324 against the first and second locking protrusions 422, 424. The combination of the rotational force provided by the user and the flexing of the second members 342 enables the user to move the ledges 344 past the first and second locking protrusions 422, 424 and into the first and second locking notches 423, 425, respectively, to lock the connector 202 and receiver 204 together.

When connector assembly 200 is in the locked state, ledges 344 of first and second locking tabs 322, 324 are each interlocked within first and second locking notches 423, 425, respectively. First and second locking notches 423, 425 therefore also block first and second locking tabs 322, 324, and hence connector 202, from being further rotated once ledges 344 of first and second locking tabs 322, 324 are engaged with first and second locking notches 423, 425, as shown in FIG. 6B. During operation, the locking mechanism enables connector assembly 200 to provide haptic feedback to the user as connector 202 is rotated to the locked state. Specifically, the flexing of the first and second locking tabs 322, 324 and urging of the ledges 344 past the first and second locking protrusions 422, 424 causes connector 202 to “snap” when the ledges 344 make contact and engage with first and second locking notches 423, 425. The haptic feedback therefore serves as an indication to the user of the successful locking of connector 202 when connecting connector 202 and receiver 204.

When ledges 344 of first and second locking tabs 322, 324 are engaged with first and second locking notches 423, 425, respectively, first and second locking tabs 322, 324 may correspondingly flex and apply a biasing force against first and second locking protrusions 422, 424 and outer surfaces 346 of the second members 342 of first and second locking tabs 322, 324. Such a biasing force assists in retaining the engagement between first and second locking tabs 322, 324 and first and second locking notches 423, 425, respectively, to keep connector assembly 200 in the locked state. When in the locked state, the rotation of connector 202 (e.g., displacement of first and second aligners 330, 332 away from first and second aligner notches 418, 420) also causes first and second aligners 330, 332 to in turn axially align with and/or contact an interior surface of receiver 204 at distal end 402. The misalignment between first and second aligners 330, 332 and first and second aligner notches 418, 420 due to the rotation of connector 202 (to lock connector 202) therefore further assists in preventing connector 202 from inadvertently disconnecting from receiver 204 when locked.

Connector 202 may correspondingly be unlocked from receiver 204 by rotating the connector 202 in the opposite direction (e.g., counter clockwise) with a force sufficient to urge the ledges 344 of the first and second locking tabs 322, 324, past the first and second locking protrusions 422, 424. Requiring a sufficient force to overcome the biasing force between first and second locking protrusions 422, 424 and the second members 342 correspondingly decreases the likelihood of inadvertently disconnecting the connector 202 due to the need for such a force to usually be intentionally applied by the user. To transition connector assembly 200 back to its unlocked state and withdraw connector 202 from receiver port 404, connector 202 can be rotated to realign the first and second aligners 330, 332 with the first and second aligner notches 418, 420 for subsequent withdrawal of proximal portion 306 from receiver port 404 thereby disconnecting connector 202 from receiver 204.

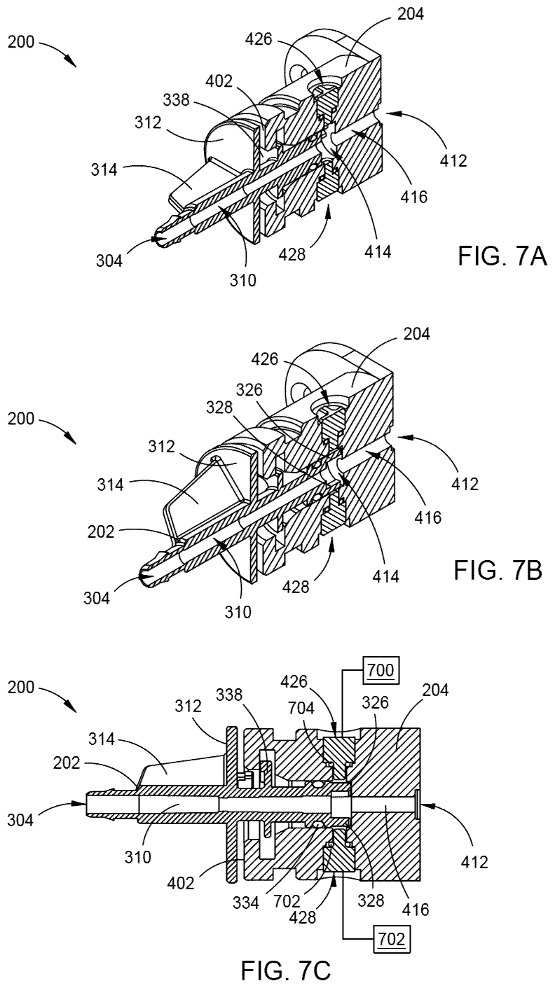

FIGS. 7A and 7B illustrate corresponding perspective cross-sectional views of connector assembly 200 as depicted in FIGS. 5B and 5C, according to certain embodiments. Meanwhile, FIG. 7C illustrates a corresponding cross-sectional side view of connector assembly 200 depicted in FIGS. 5C and 7B, according to certain embodiments. Accordingly, FIGS. 7A-7C are herein described together for clarity.

In some embodiments, the rotation of connector 202 relative to receiver 204 enables the detection of when connector assembly 200 is in the locked state. As discussed above, in some embodiments, receiver 204 may be integrated with surgical console 100. In such examples, first and second light pipe apertures 426, 428, disposed on opposite sides of housing 400, may be connected to a light pipe 700 and a sensor 702, respectively, for propagating light between first and second light pipe apertures 426, 428 and through chamber 414. In certain embodiments, light pipe 700 comprises a light emitting diode (LED), LED light pipe, a laser emitting light source, or other light source, and sensor 702 comprises a corresponding sensor or detector thereof. When in use, light from light pipe 700 may propagate from first light pipe aperture 426 towards second light pipe aperture 428 and sensor 702 connected thereto.

When connector assembly 200 is not in the locked state, the space in chamber 414 between first and second light pipe apertures 426, 428 is unobstructed, thereby allowing light from light pipe 700 to propagate through chamber 414 and be received and/or detected by sensor 702. When connector assembly 200 is in the locked state with first and second light posts 326, 328 disposed within chamber 414, first and second light posts 326, 328 are sized sufficiently to block the first and second light pipe apertures 426, 428 when connector 202 is inserted and rotated (e.g., to lock the connector 202). The blocking of the first and second light pipe apertures 426, 428 in turn obstructs the propagation of light from light pipe 700 with which such obstruction detectable by surgical console 100 via sensor 702. Surgical console 100 may thus use the detection of light from light pipe 700 extending through chamber 414 (and the corresponding lack of detection thereof when light pipe apertures 426, 428 are blocked) as a mechanism for detecting when connector 202 is fully inserted and sufficiently rotated within receiver 204 such that connector assembly 200 is transitioned to the locked state.

For example, when connector 202 is first inserted into receiver 204 and prior to connector 202 being rotated, as shown in FIGS. 5B and 6A, the first and second light pipe apertures 426, 428 remain unobstructed by the insertion of connector 202 into the chamber 414. With first and second light pipe apertures 426, 428 unobstructed, light from light pipe 700 continues to propagate through chamber 414 along with the corresponding detection thereof by surgical console 100 (e.g., continued receiving of light by sensor 702). Surgical console 100 may in turn use the continued detection of light from light pipe 700 as an indication for determining that connector 202 has not been sufficiently inserted and/or rotated to be locked with receiver 204. Such determinations by surgical console 100 indicating that connector 202 and gas supply line 114 coupled thereto may not be properly connected may prevent surgical console 100 from initiating the flow of gas from infusion gas source 430. In certain embodiments, light pipe 700 and sensor 702 may be constantly active as long as surgical console 100 is on such that light from light pipe 700 continues to propagate between first and second light pipe apertures 426, 428 up until the apertures 426, 428 become correspondingly blocked by the insertion and rotation of connector 202.

When connector 202 is rotated as shown in FIGS. 5C and 6B after being inserted into receiver port 404, the rotation of connector 202 in turn translates first and second light posts 326, 328 extending from proximal end 336 of connector tube 310 within chamber 414. As shown in FIGS. 7B and 7C, when connector 202 is sufficiently rotated such that the connector assembly 200 is in the locked state as shown in FIG. 5C, the rotation of connector 202 translates the first and second light posts 326, 328 to positions that block and obstruct the propagation of light from light pipe 700 between the first and second light pipe apertures 426, 428 and the corresponding receipt of light by sensor 702.

When sensor 702 of surgical console 100 fails to detect the propagation of light from light pipe 700, surgical console 100 may use the lack of detection of light by sensor 702 as an indication that connector 202 is inserted and sufficiently rotated to transition connector assembly 200 to the locked state. The utilization of light from light pipe 700 and corresponding detection thereof by surgical console 100 for indication of when connector 202 is inserted and locked enables surgical console 100 to also provide corresponding notifications to the user. In certain embodiments, surgical console 100 may provide visual notifications on display 120 regarding the locked state of connector 122 to assist the surgeon and/or surgical staff in confirming all necessary connectors of the surgical console 100 are properly connected and locked prior to the start of the procedure.

In some embodiments, first and second light pipe apertures 426, 428 may each also be configured with O-rings 704 to assist in creating a seal in chamber 414. As discussed above, O-ring 334 on connector tube 310 may also be disposed in chamber 414 when connector 202 is inserted into receiver 204. When connector 202 is rotated and locked thereby providing an indication to surgical console 100 via the blocking of the first and second light pipe apertures 426, 428 as discussed above, surgical console 100 may notify the user that connector 202 is properly connected and ready for the initiation of gas flow from infusion gas source 430. Due to the extension of the first and second light posts 326, 328 in chamber 414, connector tube 310 is not in direct contact with receiver tube 416. As such, when gas is delivered from infusion gas source 430, the gas flows from supply port 412 through the receiver tube 416 and chamber 414 to reach connector tube 310 and the gas supply line 114 connected to the distal port 304. The combination of O-ring 334 on connector tube 310 and O-rings 704 adjacent to the first and second light pipe apertures 426, 428 may therefore assist to maintain a seal in chamber 414 as gas flows between connector tube 310 and receiver tube 416 thereby maintaining the pressure of the gas provided to the gas supply line 114 during the surgical procedure.

In summary, embodiments of the present disclosure include structures and mechanisms for facilitating and improving surgical console setup during ophthalmic procedures, and in particular, improved port connector assemblies for intraocular fluid infusion. The connector assemblies described above include embodiments that enable temporarily locking the connector and corresponding receiver/port of the surgical console together by rotating the connector after insertion. When integrated with the surgical console, the receiver may also be configured to utilize the rotation of the connector when locking the connector to also provide a mechanism for the surgical console to detect/determine when said connector is inserted and sufficiently rotated such that the connector and receiver are properly connected and locked. Accordingly, the aforementioned connector assemblies are particularly beneficial in assisting users, such as surgeons and surgical assistants, to ensure proper connecting of supply lines for delivering infusion gases prior to surgical procedures and the maintaining of the connection throughout such procedures.

While the foregoing is directed to embodiments of the present disclosure, other and further embodiments of the disclosure may be devised without departing from the basic scope thereof, and the scope thereof is determined by the claims that follow.

What is claimed is:

1. A connector for a surgical console, comprising:
a proximal portion having a proximal port configured to be received by the surgical console;
a distal portion having a distal port;
a tube extending between the proximal portion and the distal portion, the proximal port in fluid communication with the distal port via the tube and the tube having a flow axis extending along and substantially parallel with a direction of flow between the proximal port and the distal port;
an annular flange formed along an outer surface of the tube and partitioning the proximal portion from the distal portion, the annular flange extending perpendicular to the flow axis from the outer surface of the tube; and
one or more light posts extending from the proximal port, the one or more light posts extending substantially parallel to the flow axis of the tube and configured to interact with a light pipe of the surgical console when the proximal portion of the connector is connected to the surgical console.

2. The connector of claim 1, wherein the proximal portion further comprises one or more aligners extending substantially perpendicular to the flow axis of the tube from the outer surface of the tube.

3. The connector of claim 1, wherein the proximal portion further comprises one or more locking protrusions, the locking protrusions configured to mate with the surgical console upon rotation of the connector.

4. The connector of claim 1, wherein the distal portion further comprises a pair of gripping fins.

5. The connector of claim 1, further comprising a gasket on one or both of the distal and proximal portions.

6. The connector of claim 1, wherein the distal portion further comprises a barbed collar for engaging with a flexible tube.

7. A receiver for connecting a connector to a surgical console, comprising:
a port at a distal end of the receiver opposite of a proximal end, the port configured to receive the connector and the proximal end of the receiver configured to connect to the surgical console;
a tube extending through the receiver from the port at the distal end of the receiver to the proximal end of the receiver, the tube having a flow axis extending along and substantially parallel with a direction of flow through the port and tube; and
a pair of light pipe apertures configured to receive an extension of a light pipe through the tube, the light pipe extending substantially perpendicular with the flow axis of the tube.

8. The receiver of claim 7, wherein the port further comprises one or more aligner notches at the distal end of the receiver.

9. The receiver of claim 7, further comprising one or more locking tabs formed along an interior surface of the port.

10. The receiver of claim 7, wherein the port is in fluid communication with an infusion gas or vacuum source when the proximal end is connected to the surgical console.

11. A connector assembly for a surgical console, comprising:
a receiver configured to be received by a receiver portion in the surgical console, wherein the receiver portion is in fluid communication with an infusion or vacuum gas source, and the receiver comprises:
a port at a distal end of the receiver opposite of a proximal end, the port configured to receive a connector and the proximal end configured to connect to the surgical console;
a receiver tube extending from the port at the distal end of the receiver through the receiver to the proximal end of the receiver; and
a pair of light pipe apertures configured to receive extension of a light pipe from the surgical console through the receiver tube;
a connector configured to be inserted into the receiver, the connector comprising:
a proximal portion having a proximal port configured to be received by the port of the receiver;
a distal portion having a distal port;
a connector tube extending between the proximal portion and the distal portion of the connector, the proximal port in fluid communication with the distal port via the connector tube;
an annular flange formed along an outer surface of the connector tube and partitioning the proximal portion from the distal portion; and
one or more light posts extending from the proximal port, the one or more light posts configured to obstruct the extension of the light pipe through the receiver tube when the receiver is received by the surgical console and the proximal portion of the connector is received by the receiver and rotated, and wherein when the receiver is received by the receiver portion of the surgical console and the connector is inserted into the receiver, the proximal port of the connector is in fluid communication with the infusion or vacuum gas source.

12. The connector assembly of claim 11, wherein the surgical console further comprises a light source for propagating the light pipe through the light pipe apertures and the receiver tube when the receiver is received by the receiver portion of the surgical console.

13. The connector assembly of claim 12, wherein the surgical console further comprises a sensor for detecting propagation of the light pipe through the pair of light pipe apertures and the receiver tube when the receiver is received by the receiver portion of the surgical console.

14. The connector assembly of claim 11, wherein:
the proximal portion of the connector further comprises one or more aligners extending from the outer surface of connector tube;
the port of the receiver further comprises one or more aligner notches; and
inserting the proximal portion of the connector into the port of the receiver comprises aligning the one or more aligners on the proximal portion of the connector with the one or more aligner notches at the port of the receiver.

15. The connector assembly of claim 11, wherein:
the proximal portion of the connector further comprises one or more locking tabs extending from an outer surface of the connector;
the port of the receiver further comprises one or more locking members along an interior surface of the port; and
when the proximal portion of the connector is inserted into the port of the receiver and rotated, the one or more locking tabs of the connector are configured to mate with the one or more locking members of the receiver to temporarily lock the connector and receiver together.
